(19) United States
(12) Patent Application Publication
(10) Pub. No.: US 2006/0099208 A1
(43) Pub. Date: May 11, 2006

(54) Method of potentiating the therapeutic action of monoclonal and polyclonal antibodies

(76) Inventor: Das Narasirmha Undurti, Shaker Heights, OH (US)

Correspondence Address: RAMA NATH, 2530, QUAIL RUN, LANSDALE, PA, 19446, US

(21) Appl. No.: 11/265,664

(22) Filed: Nov. 2, 2005

(30) Foreign Application Priority Data
Nov. 8, 2004 (IN) 1159/CHE/2004

(51) Int. Cl. A61K 39/395; A61K 31/202

(52) U.S. Cl. 424/133.1; 424/145.1; 514/560

(57) ABSTRACT

A method of potentiating the action of monoclonal/polyclonal antibodies against growth factors and their receptors by coupled conjugation with EFAs/PUFAs (optionally with a lymphographic agent) in the prevention and/or treatment of cancer, and inflammatory conditions such as rheumatoid arthritis, systemic lupus erythematosus, progressive systemic sclerosis, mixed connective tissue disorder, vasculitis. The disorders may be due to uncontrolled angiogenic activity causing proliferative diabetic retinopathy, other eye disorders such as macular degeneration, skin problems such as psoriasis, renal conditions such as proliferative glomerulonephritis, lymphoma and leukemias. The EFAs/PUFAs may be chosen from linoleic acid, gamma-linolenic acid, dihomo-gamma-linolenic acid, arachidonic acid, alpha-linolenic acid, eicosapentaenoic acid, docosahexaenoic acid, cis-parinaric acid, docosapentaenoic acid and conjugated linoleic acid. The method selectively reduces blood supply to a neoplastic region, such as a tumor region, by causing occlusion of blood vessels feeding the tumor region. The invention also provides solutions/salts of PUFAs, in combination with a lymphographic agent.

### FIELD OF THE INVENTION

This invention generally relates to a method and technique for stabilizing and potentiating the therapeutic actions of monoclonal and polyclonal antibodies against various growth factors and other proteins. More particularly, the invention relates to a drug composition for potentiating the therapeutic actions of monoclonal and polyclonal antibodies against various growth factors and other proteins by the use of essential fatty acids (EFAs) polyunsaturated fatty acids (PUFAs).

### BACKGROUND OF THE INVENTION

Growth factors and cancer: Growth factors are generally proteins secreted by several types of cells in the body that have potent actions on cell proliferation, migration, and differentiation. These growth factors bind to their respective receptors that in turn lead to the activation of transmembrane receptor tyrosine kinases. Growth factors that are particularly relevant to the study of cancer include epidermal growth factor (EGF), vascular endothelial growth factor (VEGF), fibroblast growth factor (FGF), platelet derived growth factor (PDGF), hepatocytes growth factor (HGF), placental growth factor (PIGF), and tyrosine kinase receptor erbB2, also known in humans as Her2. Unfortunately, these growth factors also stimulate angiogenesis that facilitates tumor growth and metastasis. Thus, desirably, inhibition of the action of these growth factors or blocking the expression of their receptors interferes with angiogenesis and suppresses tumor growth and metastasis.

These growth factors are believed to have a role in many inflammatory conditions such as rheumatoid arthritis (RA), systemic lupus erythematosus (SLE) also called as lupus, scleroderma, vasculitis, and other collagen vascular diseases; and in atherosclerosis, glomerulonephritis both in primary and secondary types of nephritis, psoriasis, and in Alzheimer's diseases, and proliferative disorders seen in diabetes mellitus (DM) such as diabetic retinopathy, vasculopathy, nephropathy, neuropathy, and atherosclerosis. In addition, it is also observed that some of the complications such as retinopathy, neuropathy, nephropathy, etc., seen in diabetes mellitus are due to enhanced production of various growth factors such as EGF, VEGF, and IGF (insulin-like growth factor in the target tissues (references 1-3).

Epidermal Growth Factor (EGF): Studies have shown that EGF stimulates cells to divide by activating members of the EGFR (EGF receptor). EGFR activation also plays an important role in cancerous tumor survival. Several types of human cancer exhibit sustained activation of EGFRs by secreted growth factors. Amplification and rearrangement of the gene encoding EGFR occur in a significant fraction of glioblastomas and squamous-cell carcinomas and correlate with reduced patient survival. Similarly, amplification of the gene encoding HER2 is associated with shorter time of breast cancer relapse, reduced overall patient survival and resistance to hormonal therapy and chemotherapy. Consistent with their pivotal role in stimulating cell proliferation, blocking EGFR function is seen to result in retarded tumor growth. Examples include the clinically approved anti-HER2 monoclonal antibody trastuzumab (Herceptin®), similar antagonists of the EGFR such as the monoclonal antibody cetuximab, and low-molecular weight inhibitors of tyrosine kinases. Erbitux® is a chimeric monoclonal antibody which is specific for the EGFR. Over expression of EGFR is common in many solid tumors, such as colorectal and lung carcinomas as well as cancers of the head and neck. It correlates with increased metastasis, decreased survival and a poor prognosis. EGFR protects malignant tumor cells from the cytotoxic effects of chemotherapy and radiotherapy, making these treatments less effective. Erbitux® binds to the extracellular domain of EGFR on the tumor cell, thereby inhibiting receptor-associated tyrosine kinase. This inhibition blocks the intracellular pathways associated with tumor cell proliferation, so preventing tumor growth and dissemination as well as inducing tumor cell death or apoptosis. There is evidence to suggest that EGF and EGFR have angiogenic actions and that Erbitux® prevents angiogenesis (references 4-6).

Vascular Endothelial Growth Factor (VEGF): VEGF is a protein that is secreted by hypoxic cells, including those that are cancerous. VEGF stimulates new blood vessel formation or angiogenesis by binding to specific receptors on nearby blood vessels to stimulate extensions of existing blood vessels. Angiogenesis plays an important role in both tumor growth and metastasis. Monoclonal antibodies are designed to bind to VEGF preventing it from binding to its receptors and therefore potentially inhibiting tumor growth. Bevacizumab is a humanized monoclonal antibody to VEGF developed by Genentech and is called as Avastin®. By inhibiting VEGF, Avastin interferes with the blood supply to tumors, a process that is critical to tumor growth and metastasis.

Several clinical studies showed that both Erbitux® and Avastin®, which are humanized monoclonal antibodies to EGFR and VEGF respectively, are useful in the treatment of colon cancer. Erbitux® shrank tumors in 22.9% of advanced colon cancer patients when combined with chemotherapy. Avastin® in combination with chemotherapy extended colon cancer patients' lives by 5 months in a trial of 900 patients.

EGF and VEGF are mentioned here only as examples on the role of various growth factors and their receptors in cancer. Several studies have indicated that blocking or neutralizing the actions of various growth factors and their receptors suppresses cancer (references 7-11).

Polyunsaturated fatty acids: The polyunsaturated fatty acids (PUFAs) are fatty acids some of which have at least two carbon-to-carbon double bonds in a hydrophobic hydrocarbon chain, which typically includes X-Y carbon atoms and terminates in a carboxylic acid group. The PUFAs are classified in accordance with a short hand nomenclature, which designates the number of carbon atoms present (chain length), the number of double bonds in the chain and the position of the double bonds nearest to the terminal methyl group. The notation “a:b” is used to denote the chain length and number of double bonds, and the notation “n:x” is used to describe the position of the double bond nearest to the methyl group. There are at least 4 independent families of PUFAs, depending on the parent fatty acid from which they are synthesized.

They include:

The “n−3” series derived from alpha-linolenic acid (ALA, 18:3, n−3).

The “n−6” series derived from cis-linoleic acid (LA, 18:2, n−6).

The “n−9” series derived from oleic acid (OA, 18:1, n−9).

The “n−7” series derived from palmitoleic acid (PA, 16:1, n−7).

It is noted that mammals cannot synthesize the parent fatty acids of the n−3 and n−6 series, and hence they are often referred to as “essential fatty acids” (EFAs). Since these compounds are necessary for normal health but cannot be synthesized by the human body, they must be obtained through proper diet (12, 13).

It is believed that both LA and ALA are metabolized by the same set of enzymes. LA is converted to gamma-linolenic acid (GLA, 18:3, n−6) by the action of the enzyme delta-6-desaturase (d-6-d) and GLA is elongated to form dihomo-GLA (DGLA, 20:3, n−6), the precursor of the 1 series of prostaglandins (PGs). DGLA can also be converted to arachidonic acid (AA, 20:4, n−6) by the action of the enzyme delta-5-desaturase (d-5-d). AA forms the precursor of 2 series of prostaglandins, thromboxanes and the 4 series of leukotrienes. ALA is converted to eicosapentaenoic acid (EPA, 20:5, n−3) by d-6-d and d-5-d. EPA forms the precursor of the 3 series of prostaglandins and the 5 series of leukotrienes. LA, GLA, DGLA, AA, ALA, EPA and docosahexaenoic acid (DHA, 22:6, n−3) are all PUFAs, but only LA and ALA are EFAs (see FIG. 1 for metabolism of essential fatty acids).

Several studies have shown that EFAs/PUFAs play a significant role in the pathobiology of inflammatory conditions such as RA, SLE, PSS; skin disorders such as psoriasis, eczema, atopic and non-atopic dermatitis; atherosclerosis, and cancer (references 13-16). Further, various PUFAs have also been shown to be of benefit in all these conditions (references 13-16).

PUFAs and cancer: Tumor cells are not only deficient in PUFAs but also have low rate(s) of lipid peroxidation, contain relatively large amounts of antioxidants such as vitamin E and superoxide dismutase (SOD). It is also believed that low rates of lipid peroxidation and consequent low amounts of lipid peroxides in the cells can contribute to an increase in the mitotic process which ultimately leads to an increase in cell proliferation. Thus, a deficiency of PUFAs, high amounts of antioxidants and the presence of low amounts of lipid peroxides in the tumor cells can contribute to the growth of tumor cells. This finding is supported by studies by the inventor wherein it was noted that PUFAs such as GLA, DGLA, AA, EPA and DHA could decrease tumor cell proliferation. In addition, it was also observed that when appropriate amounts of GLA, DGLA, AA, EPA and DHA were administered to tumor cells as well as normal cells which were obtained from American Type Culture Collection, only tumor cells were killed without having any significant action on the survival of normal cells in-vitro. In mixed culture experiments, in which both normal and tumor cells were grown together, GLA showed more selective tumoricidal action compared to AA, EPA and DHA though, these latter fatty acids were also effective to some extent. This indicated that selective delivery of GLA, DGLA, AA, EPA and DHA to tumor cells might offer a new therapeutic approach in the treatment of cancer (references 17-22).

The above referenced in-vitro results are supported by in-vivo studies performed in animal tumor models. For example, it was noted that GLA, DGLA, AA, EPA and DHA when used either in the form of pure fatty acid alone or in the form of fatty acid rich oils, could inhibit the growth of skin papilloma in mice, formation and growth of hepatoma in rats and ascitic tumor cells in the peritoneum of experimental animals. These results indicate that these fatty acids can inhibit the growth of a variety of tumors even in-vivo. In further studies, it was noted that these fatty acids are able to enhance free radical generation and the lipid peroxidation process selectively in the tumor cells but not so much in the normal cells and thus, are able to bring about their cancer killing action (references 23-28).

### SUMMARY OF THE INVENTION

Generally, the present invention teaches augmenting the efficacy of therapeutic monoclonal and polyclonal antibodies. More specifically, the invention teaches the use of PUFAs in potentiating and augmenting monoclonal and polyclonal antibodies in the prevention and/or treatment of various cancers, and inflammatory conditions such as atherosclerosis, rheumatoid arthritis (RA), systemic lupus erythematosus (SLE), progressive systemic sclerosis (PSS), mixed connective tissue disorder (MCTD), vasculitis, and other disorders including those caused by uncontrolled angiogenic activity such as proliferative diabetic retinopathy; other eye disorders such as macular degeneration; and skin problems such as psoriasis, and various forms of proliferative glomerulonephritis and other disorders in which cell proliferation and angiogenesis play a dominant role.

In a series of investigations, the inventor observed that the cytotoxic action of anti-cancer drugs such as doxorubicin, vincristine and cis-platinum can be augmented by various PUFAs such as GLA, DGLA, AA, EPA and DHA. In addition, these fatty acids could also enhance the cellular uptake of these anti-cancer drugs by the tumor cells and thus, are able to potentiate the anticancer actions of these drugs (27). Inventor's studies have shown that PUFAs can be selectively exploited as possible anti-cancer agents either alone or in combination with traditional anti-cancer drugs

It is to be noted in this context that PUFAs can bind to albumin and other proteins and hence, if given intravenously may not be available to be taken up by the tumor cells and consequently may not be able to bring about their cell killing action on the tumor cells. In view of this, it is desirable that PUFAs including GLA should be delivered to the patients in such a manner that they are easily available to the tumor (tumor cells) and are delivered selectively to the tumor cells. It is highly desirable that PUFAs including GLA be given intra-tumorally as was experimentally done in the case of human gliomas, or, intra-arterially by selective intra-arterial infusion as was done experimentally in the case of hepatoma and giant cell tumor of the bone. But, it is also possible that in some cases of cancer such as Hodgkin's and non-Hodgkin's lymphoma wherein the tumor cells are extremely sensitive to the cytotoxic actions of PUFAs, even oral administration may be sufficient as was observed in certain patients. Since, PUFAs can potentiate the cell killing effect of anti-cancer drugs and cytokines, it is desirable to administer a combination of PUFAs, anti-cancer drugs, or specific monoclonal or polyclonal antibodies to growth factors or a combination thereof so that fatty acids are carried to the tumor cells.

In one form, the invention resides in a method of potentiating therapeutic action of monoclonal and polyclonal antibodies, comprising directing said antibodies selectively against growth factors, their receptors and intra and extra-cellular proteins in the form an admixture of said antibodies conjugated selectively with one or more essential fatty acids and polyunsaturated fatty acids. A modification teaches a method of selectively delivering PUFAs that are toxic to cancer cells by conjugating them or mixing them with monoclonal antibodies or specific polyclonal antibodies to growth factors that are known to enhance tumor growth, such that tumoricidal PUFAs are delivered to the cancer cells very selectively. In addition, such a combination of PUFAs and monoclonal and/or specific polyclonal antibodies to growth factors will also inhibit blood supply to a tumor such that ultimately the tumor cells will die by administering two types of substances for example: one a lipid and the other a protein or a peptide both of which have very potent anti-angiogenic action

Available information and observations attest to the fact that malignant tumors are dependent on various growth factors for their growth and are also angiogenesis-dependent diseases. Growth factors also behave as angiogenic factors and thus, promote tumor growth. But, it should be noted here that tumor-associated angiogenesis is a complex, multi-step process which can be controlled by both positive and negative factors. It appears, as though, angiogenesis is necessary, but not sufficient, as the single event for tumor growth (29). But, it is evident from several experimental results that angiogenesis may be a common pathway for tumor growth and progression. Though several anti-angiogenic agents, antibodies to growth factors are being tried to arrest tumor growth, these are not without problems. Since the majority of these agents are proteins/peptides, their long-term use may lead to the development of antibodies, which can neutralize their action. These anti-angiogenic and anti-growth substances need to be given repeatedly and some of them are unstable and are difficult to produce in large amounts. In view of this, it is desirable and necessary to make efforts to stabilize and potentiate the actions of known anti-angiogenic molecules, and antibodies against various growth factors.

All the above factors and observations attest to the fact that both EFAs/PUFAs and growth factors have important roles in many clinical conditions. In view of the significant role of growth factors in the growth and progression of cancer including metastasis, several attempts have been made and are being made to develop specific monoclonal and polyclonal antibodies that specifically inhibit or block the actions of these growth factors. Such attempts have been found to inhibit the growth of the tumor cells. It is known that these monoclonal and polyclonal antibodies against various growth factors also inhibit angiogenesis that ultimately leads to the regression of the tumors. But, it should be mentioned here that in the majority of the instances these monoclonal and polyclonal antibodies against various growth factors have not been very effective in inhibiting the growth of various tumors. Certain preferred examples of this failure on the part of monoclonal and polyclonal antibodies developed against various growth factors to effectively prevent the growth of various tumors are: Erbitux® and Avastin®, which are humanized monoclonal antibodies to EGFR and VEGF respectively. Erbitux® shrank tumors in only 22.9% of advanced colon cancer patients when combined with chemotherapy, whereas Avastin in combination with chemotherapy extended colon cancer patients' lives only by 5 months in a trial of 900 patients. Furthermore, both Erbitux® and Avastin® have many side effects. This clearly shows that monoclonal and polyclonal antibodies developed against various growth factors are not very effective in preventing the growth of tumors and are ineffective in preventing angiogenesis. In the same manner other monoclonal and polyclonal antibodies developed against several other growth factors also failed to be effective in the treatment of other diseases such as psoriasis, inflammatory conditions such as rheumatoid arthritis (RA), systemic lupus erythematosus (SLE), progressive systemic sclerosis (PSS), mixed connective tissue disorder (MCTD), vasculitis; and other disorders caused by uncontrolled angiogenic activity such as proliferative diabetic retinopathy; other eye disorders such as macular degeneration; and skin problems such as psoriasis, various forms of proliferative glomerulonephritis, and other disorders in which cell proliferation and angiogenesis plays a dominant role. It may be mentioned here that specific monoclonal and polyclonal antibodies are also being developed against various receptors on the cell surfaces and other markers with the hope that these antibodies will be useful in the management of various clinical conditions mentioned above.

The present invention specifically teaches the efficacious use of monoclonal and polyclonal antibodies developed against various growth factors, receptors, and other cell surface and intracellular markers and proteins by coupling them to PUFAs such that the actions of these antibodies are potentiated. It has been discovered that the beneficial actions of compounds formed as a result of such coupling of monoclonal and polyclonal antibodies with PUFAs will be more than the combined individual effect observed when these antibodies and PUFAs are administered separately.

The monoclonal and polyclonal antibodies referred to herein include growth factor(s), their receptor(s), cell surface receptors or markers, and intracellular proteins that have various physiological and pathological actions. The lipid may be one or more of the PUFAs: LA, GLA, DGLA, AA, ALA, EPA and DHA.

Another embodiment resides in a combination drug comprising therapeutic monoclonal/polyclonal antibodies to growth factors and their receptors, and intra and extra-cellular proteins, said antibodies being made into an admixture and conjugated selectively with one or more essential fatty acids and polyunsaturated fatty acids. A modification of the drug resides in a drug-composition having a covalently coupled or complexed form containing a monoclonal and polyclonal antibody (dies) and one or more EFAs/PUFAs containing between 14 and 26 carbon atoms as an example. Also taught herein are pharmacological compositions comprising amides of the PUFAs combined with a monoclonal antibody or polyclonal antibody against growth factors and various proteins (either extracellular or intracellular) such that the compositions are stable enough to pass through the acidic environment of the stomach, in the blood stream, and also enter the brain crossing the blood brain barrier as desired. Also taught herein is an amide derivative of monoclonal and polyclonal antibodies against various growth factors with biological activities useful in many clinical conditions that have been enumerated above. It should be mentioned here that PUFAs are herein not used as carriers (though they may serve as carriers under certain circumstances when combined, complexed or covalently linked to/with the antibodies) but themselves serve as effective agents to treat the clinical condition in question and also potentiate the actions of various monoclonal and polyclonal antibodies.

The invention also teaches different methods of administering the combination drug to patients to obtain efficacious results. In another form, the present invention teaches the efficacious use of anti-growth factor and anti-angiogenic substances, which can inhibit endothelial cell proliferation, growth of tumor cells and coupling them to cis-unsaturated fatty acids, which also have anti-angiogenic and cytotoxic actions on tumor cells, such that the actions of these substances are potentiated by each other. Further, as angiogenesis is involved in other disease processes such as inflammation, tumor metastasis, etc., it is envisaged that the conjugate(s) of anti-angiogenic substances and PUFAs will be useful in these diseases also.

In this context, it is important to note that the inventor has found that polyunsaturated fatty acids (PUFAs) such as gamma-linolenic acid (GLA), dihomo-GLA (DGLA), arachidonic acid (AA), eicosapentaenoic acid (EPA) and docosahexaenoic acid (DHA) can selectively kill the tumor cells (as described above), and under specific conditions and in conjugation with salts such as lithium and a lymphographic agent, these fatty acids can actually behave as anti-angiogenic substances, i.e. they block all the blood supply to the tumor and also prevent generation of new blood vessels. Using these fatty acids in this particular combination, the inventor has successfully treated human hepatocellular carcinoma and giant cell tumor of bone with few or no ill effects or side effects.

Described specifically hereinafter is a novel combination of a protein and a lipid, and different methods of administering the same. The protein referred to herein is a potent and specific antagonist of growth factor(s) and inhibitor of endothelial proliferation and angiogenesis. The lipid may be one or more of the polyunsaturated fatty acids: LA (linoleic acid), GLA, DGLA, AA, ALA (alpha-linolenic acid), EPA, DHA and cis-parinaric acid. In this instance or method the polyunsaturated fatty acid need to be given only once or at the most twice within a period of 1 to 2 months. This invention teaches that unlike anti-growth factor antibodies, these fatty acids are not only cytotoxic to the tumor cells but are also able to function as anti-angiogenic agents. Further, polyunsaturated fatty acids when given in the formulated form, are more potent than mere monoclonal antibodies or polyclonal antibodies per se against growth factors in their anti-angiogenic and anti-cancer actions.

The invention in another aspect teaches a method of killing tumor cells by using a combination of monoclonal antibodies/polyclonal antibodies against growth factors that have been conjugated to polyunsaturated fatty acids to cause necrosis or apoptosis of tumor cells. The invention also provides a method of causing anti-angiogenic action in the tumor region with the result that new blood vessels and collaterals are not formed to sustain the tumor. The present invention in another aspect addresses the issue of drug delivery to the target tissue and provides the most efficacious method of administering an admixture of selected PUFAs with other elements such as anti-growth factor antibodies as will be described hereinafter.

The invention in yet another aspect teaches a method of regressing the tumor/or interrupting blood supply to the tumor using a pre-determined admixture of at least a PUFA and an anti-growth factor antibody to produce necrosis with very desirable results. Both the PUFAs and anti-growth factor antibodies being similar in function, the invention also provides a method of causing anti-tumor and anti-angiogenic action in the tumor region with the result that new blood vessels and collaterals are not formed to sustain the tumor in the tumor region treated according to the invention. Also described herein are examples of efficacious methods of administering an admixture of selected PUFAs along with an anti-growth factor antibody as will be described in detail hereinafter.

### BRIEF DESCRIPTION OF THE DRAWING

A more detailed understanding of the invention may be had from the following description of embodiments, given by way of example and to be understood in conjunction with the accompanying drawing wherein:

FIG. 1 illustrates metabolism of essential fatty acids.

### DESCRIPTION OF EMBODIMENTS

In the following detailed description of the various embodiments of the invention, reference is made to examples through which the invention may be practiced. These embodiments are described in sufficient detail to enable those skilled in the art to practice the invention, and it is to be understood that other embodiments may be utilized and that changes may be made without departing from the scope of the present invention. The following detailed description is therefore not to be taken in a limiting sense, and the scope of the present invention is defined only by the appended claims and their equivalents.

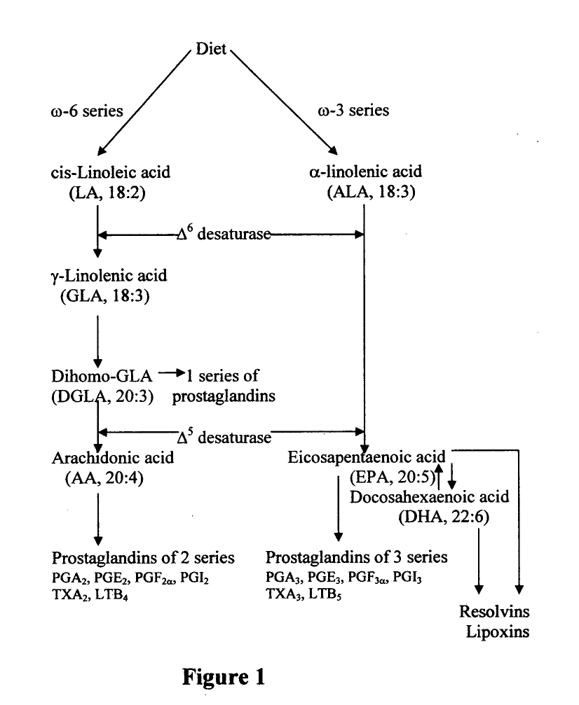

With specific reference to FIG. 1, the metabolism of essential fatty acids and their independent therapeutical benefits are understood from the description herein. The use of EFAs and PUFAs in general in the context of this invention are described in the following paragraphs.

Modification of monoclonal and polyclonal antibodies with EFAs/PUFAs/LCPUFAs: For potentiating the action of therapeutic monoclonal and polyclonal antibodies, an exemplary method is to administer an admixture obtained by mixing or conjugating specific monoclonal and polyclonal antibodies such as Erbitux® and Avastin® with EFAs/PUFAs such that they form stable complexes. The conjugation between monoclonal antibodies of EGFR and VEGF and EFAs/PUFAs can be a covalent bond, preferably an amide bond, which survives the conditions in the stomach if the admixture is given parenterally. Such anti-EGFR antibody-EFAs/PUFAs complex and anti-VEGF antibody-EFAs/PUFAs complex will be stable without interfering with the actions of monoclonal antibodies of EGFR and VEGF such that they will be able to suppress tumor cell proliferation, tumor metastasis and angiogenesis.

The above anti-EGFR antibody-EFAs/PUFAs and anti-VEGF antibody-EFAs/PUFAs complexes can be given orally, parenterally (including but not limited to subcutaneous, intravenous, intra-arterial, rectal, submucosal) and as aerosols for administration through nose, mouth and intra-tracheal routes. These complexes and preparations could alternatively be given also rectally.

It is noted that these anti-EGFR antibody-EFAs/PUFAs and anti-VEGF antibody-EFAs/PUFAs complexes will have significant inhibitory action on tumor cell proliferation and inhibit angiogenesis and also occlude specifically tumor feeding blood vessels irrespective of the route/method of administration.

The ratio between anti-EGFR antibody and EFAs/PUFAs can vary from 1:1 to 1:1000 and 1:1 to 1000:1. The anti-EGFR and anti-VEGF antibodies can be conjugated with any one or a combination of fatty acids. For example, anti-EGFR antibody can be conjugated with LA, GLA, DGLA, AA, ALA, EPA and/or DHA and similarly anti-VEGF antibody is conjugated with LA, GLA, DGLA, AA, ALA, EPA and/or DHA. In certain instances, EFAs/PUFAs may be conjugated with both anti-EGFR and anti-VEGF antibodies simultaneously. The EFAs/PUFAs can be in the form of pure acid, sodium salt, lithium salt, meglumine salt, magnesium salt, iodized salt and/or any other type of stable salt. For intra-arterial injection the preferred conjugate is in the form of an amide or complex between Li-LA, Li-GLA, Li-DGLA, Li-AA, Li-ALA, Li-EPA or Li-DHA and iodized oily lymphographic oil such as Lipiodol, and anti-EGFR antibody and/or anti-VEGF antibody.

The amount of these anti-EGFR and anti-VEGF antibodies to be given orally or parenterally can vary from 1 mg per dose to 100 gm. These anti-EGFR antibody-EFAs/PUFAs complexes can be given daily as a single injection or as a continuous infusion in a day and/or daily for a period of one week or as frequently as needed depending on the response. Administration of these complexes can be repeated daily, weekly or monthly as the situation demands.

In the same fashion as described for anti-EGF and anti-VEGF antibodies and their conjugation with various EFAs/PUFAs, similar complexes can also be prepared between anti-TNF-α, anti-IL-1, anti-IL-2, anti-IL-6, etc., antibodies and various EFAs/PUFAs.

The compounds of the invention containing a monoclonal and polyclonal antibody (dies) against a growth factor(s) and its receptor(s), and various intra and extracellular proteins and one or more of PUFAs can be prepared in pharmaceutical preparations containing the compounds themselves or their selected derivatives in appropriate or suitable proportions. Administration may be made by any method, which allows the compound (containing the monoclonal and polyclonal antibody (dies) and one or more of PUFAs) to reach the site of desired action including the brain. The compound(s) can be administered orally in the form of dragees, tablets, and syrups or by inhalation or ampules. When compounds are administered rectally, the composition can be in the form of a suppository. When the compounds of the invention are to be administered by topical application for instance for various skin conditions, they can be in the form of pomade or a gel. Another example of administration can be as an intra-tumoral preparation in appropriate doses for the treatment of human brain gliomas or any other accessible tumor (eg. urinary bladder cancer, carcinoma of the esophagus, carcinoma of the lung, breast cancer, etc.) by any route by using flexible fiber optic scopes such as bronchoscope, etc. Another method of administration of the preparation can be in the form of selective intra-arterial infusion or injection into the tumor-feeding vessel per se by femoral, brachial or carotid routes or any other suitable route or in a combination of routes or any other suitable agent, all in a mixture or in conjugated form(s) (like GLA or lithium GLA+monoclonal and polyclonal antibody (dies), LA/GLA/DGLA/AA/ALA/EPA/DHA/cis-parinaric acid/docosapentaenoic acid or their salts including lithium salts all individually or in combination thereof together with monoclonal and polyclonal antibody (dies), LA/GLA/DGLA/AA/ALA/EPA/DHA/cis-parinaric acid/docosapentaenoic acid or their salts including lithium salt in combination with or conjugated to monoclonal and polyclonal antibody (dies), which show selective occlusion of tumor feeding vessels and/or anti-angiogenic actions and anti-cancer action. Further, the compound(s) can be delivered using suitable devices, or a slow releasing capsule/tablet at an appropriate site or organ of the body. This preparation can be administered daily, weekly, or monthly or at various intervals as deemed appropriate. This preparation can be given as an intravenous infusion continuously or intermittently in a day, daily, weekly or monthly or several times a day or as frequently as necessary depending on the necessity.

It is also within the purview of this invention, as stated supra to administer an admixture of PUFAs, anti-cancer drugs, and selected anti-growth factor, and anti-angiogenic substance(s), at the same time administering predetermined doses of PUFAs orally. All such variations are envisaged to be within the ambit of this invention.

Application to mammals: Even though the examples described supra relate to humans, it is envisaged that the method of suppressing or reducing the growth of the cancer and/or anti-angiogenic action using the admixture of this invention including an anti-growth factor(s)/anti-cytokine(s) antibodies and/or angiogenic substances are equally applicable to other mammals. Likewise, the combination drug and the pharmaceutical composition described may be administered to mammals in general.

While this invention has been particularly shown and described with reference to exemplary embodiments thereof, it will be understood by those skilled in the art that various changes in form and details may be made therein without departing from the crux of the invention. For example, the exemplary monoclonal and polyclonal antibodies stated herein above comprise Erbitux® and Avastin® but might include others as well. The anti-growth factors referred to herein as examples comprise EGF and VEGF but might include other agents with actions on cancer cells and angiogenesis. Also, sodium and potassium salts are considered equivalents of each other. Many other embodiments within the ambit of the invention will be apparent to those skilled in the art. Those skilled in the art will recognize or be able to ascertain that there are many equivalents to the specific embodiments of the invention described specifically herein. All such equivalents are intended to be encompassed in the ambit and scope of the appended claims and their equivalents.

1. A method of potentiating therapeutic action of monoclonal and polyclonal antibodies, comprising:
directing said monoclonal and polyclonal antibodies selectively against growth factors, their receptors and intra and extra-cellular proteins in the form an admixture of said antibodies conjugated selectively with one or more essential fatty acids (EFAs) and polyunsaturated fatty acids (PUFAs).

2. The method as in claim 1, wherein said one or more essential fatty acids and polyunsaturated fatty acids have molecules containing 18 to 22 carbon atoms.

3. The method as in claim 1, wherein said growth factors, their receptors and intra and extra-cellular proteins comprise EGF (epidermal growth factor), VEGF (vascular endothelial growth factor), fibroblast growth factor (FGF), platelet derived growth factor (PDGF), hepatocytes growth factor (HGF), placental growth factor (PIGF), and tyrosine kinase receptor erbB2 which is otherwise known in humans as Her2.

4. The method as in claim 3, wherein said one or more essential fatty acids and polyunsaturated fatty acids contain molecules selected from the group consisting of C18:2, C18:3, C20:3, C20:4, C20:5, C22:5, C22:6, cis-parinaric acid, conjugated linoleic acid.

5. The method as in claim 4, wherein said EFAs and PUFAs have at least two carbon-to-carbon double bonds in a hydrophobic hydrocarbon chain, and wherein said conjugating is done to include formation of a salt selected from the group consisting of a lithium salt, a sodium salt, a potassium salt, a magnesium salt, a calcium salt, a manganese salt, aft iron salt, a copper salt, an aluminum salt, a zinc salt, a chromium salt, a cobalt salt, a nickel salt and an iodide.

6. The method as in claim 4, wherein said conjugating is done to include a fatty acid derivative selected from the group consisting of glycerides, esters, free acids, amides, phospholipids and salts, for use in treating proliferative disorders associated with angiogenesis, including malignant tumors.

7. A combination drug comprising:
therapeutic monoclonal/polyclonal antibodies against growth factors and their receptors, and intra and extra-cellular proteins, said antibodies being made into an admixture and conjugated selectively with one or more fatty acids selected from EFAs and PUFAs.

8. The drug as in claim 7, wherein said one or more essential fatty acids and polyunsaturated fatty acids have molecules containing 18 to 22 carbon atoms, for treatment of a malignant tumor wherein the wherein the malignant tumor is cancer selected from hepatoma, bronchogenic cancer of the lung, colon cancer, breast cancer, ovarian cancer, cancer of the kidney, skin cancer, Kaposi's sarcoma, cancer of the esophagus, cancer of the stomach, leukemias, lymphomas, multiple myeloma.

9. The drug as in claim 7, wherein said EFAs and PUFAs have at least two carbon-to-carbon double bonds in a hydrophobic hydrocarbon chain, said drug for treatment of a malignant tumor wherein the tumor is cancerous and is selected from hepatoma, bronchogenic cancer of the lung, colon cancer, breast cancer, ovarian cancer, cancer of the kidney, skin cancer, Kaposi's sarcoma, cancer of the esophagus, cancer of the stomach, leukemias, lymphomas, multiple myeloma.

10. The drug as in claim 7, wherein said growth factors, their receptors and intra and extra-cellular proteins comprise EGF (epidermal growth factor), VEGF (vascular endothelial growth factor), fibroblast growth factor (FGF), platelet derived growth factor (PDGF), hepatocytes growth factor (HGF), placental growth factor (PIGF), and tyrosine kinase receptor erbB2 which is otherwise known in humans as Her2.

11. The drug as in claim 7, including a pharmaceutically acceptable carrier for administration by one or more of: oral intake, inhalation, injection and continuous fusion.

12. The drug as in claim 11, wherein a weight ratio of said antibodies to fatty acid in the composition and the weight ratio of antibodies to growth factor and/or its receptor to fatty acid ranges from 1:10 to 10:1 respectively.

13. A drug comprising,
monoclonal/specific polyclonal antibodies against growth factors and/or their specific receptors conjugated by covalently coupling to a straight-chained fatty acid molecule,
wherein the antibodies are directed selectively against growth factors and their receptors selected from the group consisting of EGF (epidermal growth factor), VEGF (vascular endothelial growth factor), fibroblast growth factor (FGF), platelet derived growth factor (PDGF), hepatocytes growth factor (HGF), placental growth factor (PIGF), and tyrosine kinase receptor erbB2, also known in humans as Her2,
wherein said straight chained fatty acid molecule is selected from the group consisting of C14:1, C16:1, C18:1, C18:2, C18:3, C20:3, C:20:4, C20:5, C22:5, C22:6, cis-parinaric acid, and conjugated linoleic acid.

14. The drug as in claim 13, wherein the straight chained fatty acid comprises a salt compound of one or more selections from the group consisting of a lithium salt, a sodium salt, a potassium salt, a magnesium salt, a calcium salt, a manganese salt, an iron salt, a copper salt, an aluminum salt, a zinc salt, a chromium salt, a cobalt salt, a nickel salt and an iodide and/or in the form of a fatty acid derivative selected from the group consisting of glycerides, esters, free acids, amides, phospholipids and salts.

15. The drug of claim 14 in the form of a pharmaceutical preparation included in a pharmaceutically acceptable carrier for treatment of diseases associated with inflammation comprising one or more of rheumatoid arthritis, lupus, progressive systemic sclerosis, mixed connective tissue disease, vasculitis, proliferative glomerulonephritis, psoriasis, diabetic retinopathy, and macular degeneration.

16. The drug of claim 14 in the form of a pharmaceutical preparation included in a pharmaceutically acceptable carrier for treatment of a malignant tumor wherein the tumor is cancerous and is selected from hepatoma, bronchogenic cancer of the lung, colon cancer, breast cancer, ovarian cancer, cancer of the kidney, skin cancer, Kaposi's sarcoma, cancer of the esophagus, cancer of the stomach, leukemias, lymphomas, multiple myeloma.

17. The drug of claim 13, for use as an oral and parenteral composition wherein a weight ratio of said antibody to said fatty acid in the composition, and a weight ratio of antibody to growth factor and/or its receptor to fatty acid ranges from 1:10 to 10:1.

18. The drug of claim 13, for use as an oral composition, wherein a quantity of said antibody to growth factor(s) and/or growth factor receptor(s) varies from 0.5 mg to 500 gm and that of said fatty acid ranges from 0.5 mg to 500 gm.

19. The drug of claim 16, prepared for administration as one of: injection subcutaneously, intravenously, intramuscularly or intra-arterially, and additionally comprising an osmolyte and prepared in a buffer at a pH value ranging from 5 to 8.

20. The drug as in claim 14, including a pharmaceutically acceptable carrier for treating proliferative diseases associated with angiogenesis.
